(19) United States
(12) Patent Application Publication
(10) Pub. No.: US 2024/0018556 A1
(43) Pub. Date: Jan. 18, 2024

(54) ACID TOLERANT CLOSTRIDIA

(71) Applicant: Alliance for Sustainable Energy, LLC, Golden, CO (US)

(72) Inventors: Jeffrey George LINGER, Denver, CO (US); Christine Ann SINGER, Lakewood, CO (US); Lauren Ann Riley DRUMM, Superior, CO (US); Michael T. GUARNIERI, Denver, CO (US); Davinia SALVACHÚA RODRÍGUEZ, Golden, CO (US); Colin Matthew KNEUCKER, Fort Collins, CO (US); Violeta SANCHEZ NOGUE, Broomfield, CO (US)

(21) Appl. No.: 18/353,673

(22) Filed: Jul. 17, 2023

Related U.S. Application Data
(60) Provisional application No. 63/389,432, filed on Jul. 15, 2022.

(51) Int. Cl. C12P 7/52; C12N 9/88; C12N 9/06

(52) U.S. Cl. CPC C12P 7/52; C12N 9/88; C12Y 401/01018; C12N 9/0014; C12Y 104/99

(57) ABSTRACT

Clostridium sp. are an obligate anaerobic bacterium that produces butyric acid as its major end-product. Previously, we have developed an integrated process that couples fermentation of biomass sugars, with in situ product recovery and distillation. This process works best at pH's lower than 7.0. The process serves to extract carboxylic acids during fermentation which enables reduced base-loading and reduced end-product toxicity. Disclosed herein are methods and non-naturally Clostridium sp. that are capable of increased production of butyric acid and increased growth at low pH conditions (e.g., pH<5.0).

### CROSS-REFERENCE TO RELATED APPLICATION

This application claims the benefit of U.S. Patent Application No. 63/389,432 filed on 15 Jul. 2022 which is incorporated by reference herein its entirety.

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

Butyric acid is most commonly produced from petroleum-derived routes. Biological routes are loaded with expenses including neutralization. Acid tolerant butyric acid production organisms can alleviate some of these burdens.

### SUMMARY

In an aspect, disclosed herein are non-naturally occurring Clostridium tyrobutyricum organisms capable of increased growth at acidic pHs and increased production of butyric acid when compared to naturally occurring C. tyrobutyricum organisms.

Other objects, advantages, and novel features of the present invention will become apparent from the following detailed description of the invention when considered in conjunction with the accompanying drawings.

### BRIEF DESCRIPTION OF THE DRAWINGS

FIGS. 1a, 1b, and 1c depict growth of wild type C. tyrobutyricum and the 11 mutagen isolates in RCM at pH 6.5 (a), pH 5.0 (b), and pH 4.5 (c). Each strain was grown for 54 hours, and samples were periodically taken.

FIGS. 2a, 2b, 2c depict growth of C. tyrobutyricum strains expressing amino acid decarboxylases with the empty vector control at pH 6.5, 5, 4.5.

FIGS. 3a, 3b, 3c depict fermentation performance from wild type Clostridium tyrobutyricum (WT ATCC25795) and chemically induced adaptive laboratory evolved Clostridium tyrobutyricum strains ALE 4.06, ALE 4.07, ALE 5.01, and ALE 5.04 in growth media at pH 6.0. FIG. 3a depicts butyric acid productivity (g/L/h) of the strains. FIG. 3b depicts glucose utilization of the strains. FIG. 3c depicts xylose utilization of the strains.

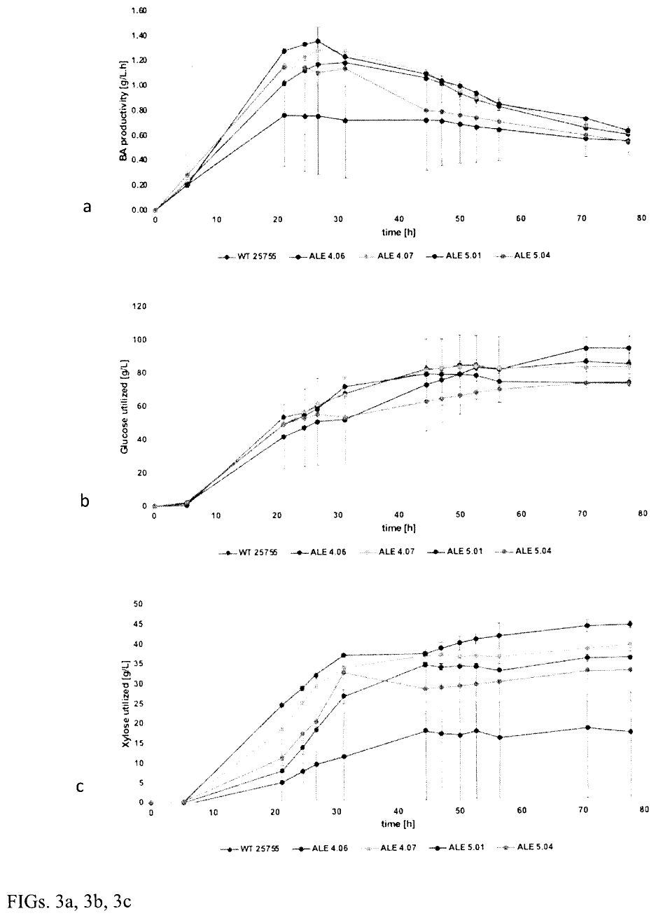

### DETAILED DESCRIPTION

In order to efficiently separate butyric acid (and other carboxylic acids) from fermentation broths in situ, the pH needs to be below the biological optimum of the favored microorganisms. Given that the chemistry here is inflexible, it is requisite to improve the acid tolerance of the microbe. We leveraged three approaches to achieve this.

For chemical mutagenesis, a mutagenized library of adaptive laboratory evolved strains was created in C. tyrobutyricum, targeting each base in the genome, using the chemical N-methyl-N′-nitro-N-nitrosoguanidine (NTG). The mutagen library and wild type C. tyrobutyricum were selected on reinforced clostridial media (RCM) plates at pH 6.5, 5.0, and 4.5. Wild type C. tyrobutyricum did not grow on the pH 5.0 and 4.5 plates while 8 colonies grew on the pH 5.0 plates and 3 colonies grew on the pH 4.5 plates for the mutagen library. The adaptive laboratory evolved strains have improved performance in low pH conditions. In an embodiment, adaptive laboratory evolved strains ALE 4.06, ALE 4.07, ALE 5.01, and ALE 5.04 were generated from chemical mutagenesis of wild type Clostridium tyrobutyricum (WT ATCC25795). In an embodiment, strain ALE 4.06 contains mutations in Clostridium tyrobutyricum (WT ATCC25795) genes including, but not limited to CTK_RS02840, CTK_RS02785, CTK_RS09915, CTK_RS08580, CTK_RS08585, CTK_RS13265, GABEKIKG_03101, CTK_RS11850, CTK_RS11005, GABEKIKG_01456, spoVD_3, and CTK_RS10285. In an embodiment, strain ALE 4.07 contains mutations in Clostridium tyrobutyricum (WT ATCC25795) genes including, but not limited to CTK_RS02860, CTK_RS09915, CTK_RS10080, argJ, GABEKIKG_02925, CTK_RS13265, GABEKIKG_03101, CTK_RS11850, CTK_RS11540, GABEKIKG_01456, and CTK_RS10285. In an embodiment, strain ALE 5.01 contains mutations in Clostridium tyrobutyricum (WT ATCC25795) genes including, but not limited to CTK_RS02840, mob, CTK_RS09785, CTK_RS09915, CTK_RS08710, CTK_RS08580, CTK_RS08585, CTK_RS07220, CTK_RS14515, CTK_RS00560, CTK_RS00210, GABEKIKG_03101, GABEKIKG_00933, apbE, GABEKIKG_01456, spoVD_3, CTK_RS00650, and CTK_RS10290. In an embodiment, strain ALE 5.04 contains mutations in Clostridium tyrobutyricum (WT ATCC25795) genes including, but not limited to CTK_RS09915, CTK_RS08580, CTK_RS08585, CTK_RS00560, CTK_RS13265, CTK_RS11850, GABEKIKG_01456, spoVD_3, and CTK_RS10285.

For targeted engineering, we focused on expression of amino acid decarboxylases due to their ability to protonate an amino acid and increase the intracellular pH. Four genes were chosen: the native lysine decarboxylase from C. tyrobutyricum, a lysine decarboxylase from Clostridium celluvorans, an agmatine deaminase from Clostridium beijerinckii, and a glutamine decarboxylase from Clostridium perfringens. Each gene was expressed using the native thiolase promoter on a plasmid in wild type C. tyrobutyricum. Each of the strains demonstrated similar growth to wild type at pH 6.5 and 5.0. At pH 4.5 all of the amino acid decarboxylases and three of the mutant isolates demonstrated enhanced growth in comparison to wild type.

Lastly, wild-type C. tyrobutyricum was subjected to adaptive laboratory evolution in a fully controlled bioreactor under anaerobic conditions and using sequential batch transfers for a cumulative 396 generations. Bacteria were cultivated at pH 5 in a sugar mixture of glucose and xylose, and transfers were performed before cells reached the stationary phase. Initial population evaluation showed an increased butyric acid productivity of 51% compared to the wild type (0.48 vs 0.32 g/L/h, respectively). Single clone isolation was performed on solid agar plates under anerobic conditions. Clone-level evaluation in glucose liquid media also showed improved butyric acid productivity compared to the wild type strain.

Materials and Methods

C. tyrobutyricum Growth Conditions

Clostridium tyrobutyricum was grown in either YTF (16 g/L Tryptone, 10 g/L yeast extract, 3 g/L sodium chloride, 5 g/L fructose, 1.0 g/L L-cysteine) or Reinforced Clostridial Medium (RCM) (10 g/L peptone, 10 g/L beef extract, 3 g/L yeast extract, 5 g/L dextrose, 5 g/L sodium chloride, 1 g/L L-cysteine, 3 g/L sodium acetate). Media was supplemented with 1.5% agar for plates and 15 μg/mL of thiamphenicol to maintain plasmids. Strains were grown at 37° C. in a Coy Anaerobic chamber or serum bottles with an atmosphere of 5% Hydrogen and 95% Nitrogen.

Chemical Mutagenesis

Chemical mutagenesis of C. tyrobutyricum was performed in the anaerobic chamber using N-methyl-N′-nitro-N-nitrosoguanidine (NTG). A 5 mg/mL NTG stock solution as prepared in citrate/phosphate buffer, pH 5.0. A 5% sodium thiosulfate solution was prepared to neutralize NTG. All lab surfaces and waste from chemical mutagenesis was subjected to sodium thiosulfate for 30 minutes prior to disposal.

Initially a kill curve was performed to determine the concentration of NTG to use for chemical mutagen library preparation. C. tyrobutyricum was grown in YTF to OD 0.5 and 1 mL of culture was aliquoted into six Eppendorf tubes. NTG was added to each tube at 0, 2, 5, 10, 15, 20 μL per mL. Cells were then incubated at room temperature for 45 minutes and cells were spun down at 7,000 rpm for 1 minute. Cell pellets were washed twice with 1 mL YTF and resuspended in 1 mL YTF. Each condition was diluted 1×10³, 10⁴, and 10⁵. On YTF agar plates, 100 μl of each dilution was plated, in duplicate. The plates were incubated overnight and colonies were counted.

A chemical mutagen library was created similar to the kill curve. Six aliquots of cell culture were subjected to 10 μL per mL NTG. Each tube was plated on 10 YTF agar plates and incubated overnight. Colonies were collected with YTF into a single tube and glycerol stocks were created and stored at −80 C.

Chemical Mutagenesis Low pH Selection

A chemical mutagenesis library glycerol stock was thawed. Cells were diluted on RCM pH 6.5, 5.0, and 4.5 X-quadrant petri plates, with each quadrant containing 100, 10, 1, and 0.1 μL of cells. Plates were incubated overnight and colonies from the pH 5.0 and 4.5 plates were streaked on pH 5.0 plates. Colonies from the streaked plates were picked into liquid and tested for acid tolerance.

Overexpression of Amino Acid Decarboxylases in C. tyrobutyricum

Four genes were inserted into the MCS of pMTL82151: the native lysine decarboxylase from C. tyrobutyricum (CTK_RS05660), a lysine decarboxylase from Clostridium celluvorans (CLOCEL_RS05175), an agmatine deaminase from Clostridium beijerinckii (CBEI_RS09960), and a glutamine decarboxylase from Clostridium perfringens (CPF_RS11275), creating pLAR208, pLAR217, pLAR219, and pLAR214 respectively. The heterologous genes were synthesized by Twist Bioscience with the native C. tyrobutyricum thiolase promoter in pMTL82151. The native gene was PCR amplified from the C. tyrobutyricum genome and Gibson assembly was used to insert the thiolase promoter and gene into pMTL82151. Each plasmid was conjugated into C. tyrobutyricum.

C. tyrobutyricum Conjugation

C. tyrobutyricum conjugation as performed using a conjugative E. coli host expressing methyltransferases, under the pBad promoter, to mimic the native C. tyrobutyricum methylome, strain AG 6805 (WM6029(5) PhiC31-R4::CTK_C23910; BL3_CLJU_c08460; BXB1_CTK_RS15605-CTK_RS13390). Each plasmid was electroporated into AG 6805 to create the conjugation donor strains. C. tyrobutyricum was grown to an OD ˜2.0 and the E. coli strains were grown to an OD of ˜1.0 with 1 mM arabinose and 1 mM Diaminopimelic acid (DAP). E. coli strains were brought into the anaerobic chamber and 1 mL of culture was spun down in microcentrifuge tubes at 7,000 RPM. Supernatant was decanted and cells were washed with 1 mL YTF. Cells were spun down again and resuspended in 1 mL YTF. 500 mL of C. tyrobutyricum was added to each tube and 500 mL of each tube was plated on YTF plates with arabinose and DAP. The plates were incubated for 24 hours. Cells were collected from each plate in 1 mL YTF. ˜100 μL of cells were plated within the agar YTF plates with thiamphenicol. Plates were incubated for 48-72 hours for colonies to form. Colonies were picked into liquid YTF with thiamphenicol, and PCR screened for the presence of the plasmid.

Testing of Acid Tolerant Strains

C. tyrobutyricum strains were tested for improved growth in low pH media in comparison to wild type in serum bottles. Serum bottles contained 50 mL of RCM at pH 4.5, 5.0, or 6.5. Precultures for each strain were grown in RCM pH 5.0 until an OD ˜1-2. Serum bottles were inoculated in duplicate to an OD of 0.05 and grown for 48 hours, shaking at 100 RPM and 37 C. 1 mL samples were taken and OD was determined.

Integration of Amino Acid Decarboxylases into the Chromosome

The three heterologous amino acid decarboxylases were integrated into the Type I Restriction Enzyme locus using CRISPR tools previously reported. First, the genes were inserted into pMTV725 between the homology arms with the native nifJ promoter using Gibson assembly. The plasmid, pMTV725, is pHW12-Plac-34RMI with p1194 driving upp added to the plasmid backbone. Each of the plasmids were conjugated into a Δupp strain of C. tyrobutyricum. Colonies were picked from the conjugation plates into YTF with thiamphenicol and grown overnight. Cells were then plated on YTF X-quadrant plates with thiamphenicol and 40 mM lactose and incubated for 48-72 hours. Colonies were picked from the plates and screened for the insertion. Cells with the insertion were plated on 5 μg/mL 5-fluorouricil and YTF X-quadrant plates to cure the strain of the plasmid. Strains were created: Δupp ΔCTK_C27600::pnifJ-C. perfingenes glutamate decarboxylase, Δupp ΔCTK_C27600::pnifJ-C. celluvorans lysine decarboxylase, and Δupp ΔCTK_C27600::pnifJ-C. beijerinkii agmatine deaminase.

Results

Chemical Mutagenesis of C. tyrobutyricum for Low pH Tolerance

Clostridium tyrobutyricum was engineered for improved growth at low pH using two methods: chemical mutagenesis and rational expression of amino acid decarboxylases. For chemical mutagenesis, a mutagen library was created in C. tyrobutyricum, targeting each base in the genome, using the chemical N-methyl-N′-nitro-N-nitrosoguanidine (NTG). Initially a kill curve was performed to determine the concentration of NTG needed for a 90% decrease in cell viability. In an embodiment, C. tyrobutyricum was exposed to 0, 5, 25, 50, 75, 100 μg of NTG for 45 minutes and cell viability was determined. The ideal NTG amount was determined to be 50 μg per mL of cells and chemical mutagenesis was performed to target each base in the genome.

In an embodiment, a kill curve of C. tyrobutyricum subjected to NTG is determined. The number of colony forming units (CFU) per mL of cells was plotted against the concentration of NTG.

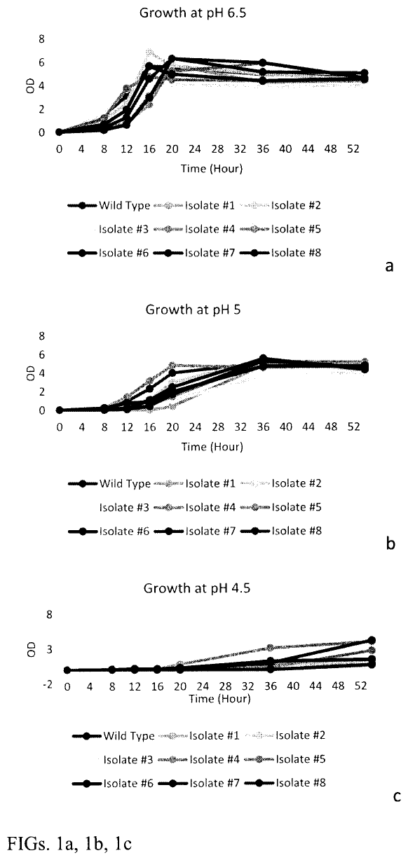

The mutagen library and wild type C. tyrobutyricum were selected on RCM plates at pH 6.5, 5.0, and 4.5. Wild type C. tyrobutyricum did not grow on the pH 5.0 and 4.5 plates while 8 colonies grew on the pH 5.0 plates (Isolate #1-8) and 3 colonies grew on the pH 4.5 plates (Isolate #9-11) for the mutagen library. The mutagen isolates were evaluated for increased growth at low pH in comparison to the wild type. Each strain was grown, in duplicate, in 50 mL RCM serum bottles at pH 6.5, 5.0, and 4.5. Samples were taken and the OD was determined. Growth curves were not determined for Isolate #9, 10, 11 due to inconsistent or no growth. Each of the strains demonstrated similar growth to wild type at pH 6.5 and 5.0 (FIG. 1a, 1b). At pH 6.5 max OD was reached at 16 hours and at pH 5.0 max OD was between 20- and 36-hour time points. At pH 4.5 three of the mutant isolates, #4, 5, and 8, demonstrated enhanced growth in comparison to wild type, reaching an OD of ˜4 for Isolate #4 and #8 and an OD of 2.75 for Isolate #5 (FIG. 1c). The 11 isolates are currently undergoing genome sequencing to determine the mutations responsible for improved growth.

Amino Acid Decarboxylase Expression

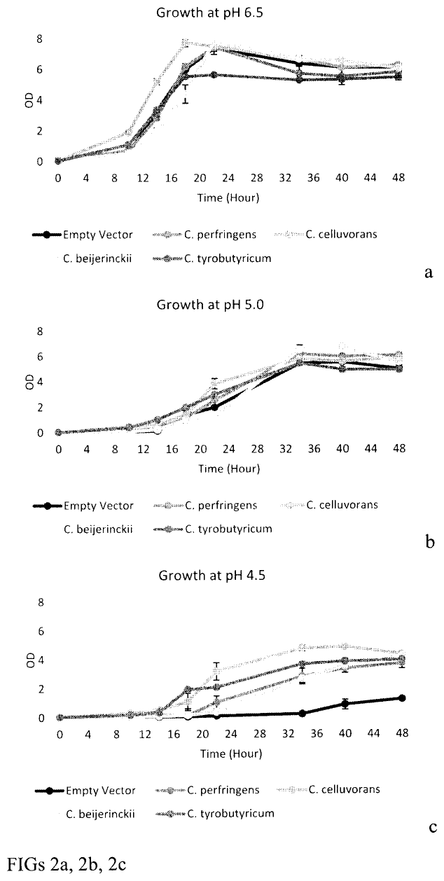

For rational expression of genes, amino acid decarboxylases were utilized due to their ability to protonate an amino acid and increase the pH within the cell. Four genes were chosen: the native lysine decarboxylase from C. tyrobutyricum, a lysine decarboxylase from Clostridium celluvorans, an agmatine deaminase from Clostridium beijerinckii, and a glutamine decarboxylase from Clostridium perfringens. The C. celluvorans and C. beijerinckii genes were previously shown to increase low pH tolerance in C. celluovrans. The C. perfringens gene has been characterized in literature to aid in growth during low pH conditions. Each gene was expressed using the native thiolase promoter on a plasmid in wild type C. tyrobutyricum. The strains were characterized in the same way as the chemical mutagen isolates. Growth at pH 6.5 and 5 was similar in all strains, reaching a max OD at 16 hours for pH 6.5 and 32 hours for pH 5 (FIG. 2a, 2b). Each of the engineered strains demonstrated increased growth at pH 4.5 in comparison to the empty vector, reaching a max OD at 32-48 hours depending on the strain (FIG. 2c). The strain expressing the C. celluvorans gene preformed the best, reaching max OD first at pH 4.5 and growing similarly in pH 5.0 and 4.5. The max OD decreases as the pH decreases, from ˜7 at pH 6.5 to ˜6 at pH 5 to ˜5 at pH 4.5.

Integration of Amino Acid Decarboxylases into the C. tyrobutyricum Genome

Each of the plasmid based amino acid decarboxylases enabled increased low pH tolerance in comparison to wild type, therefore they were integrated into the chromosome for stable expression. The CRISPR system previously described in C. tyrobutyricum was utilized to integrate each into the Type I Restriction Enzyme locus. Due to issues with thiolase promoter stability and expression, another strong native promoter was utilized, PnifJ. Each gene was integrated into the chromosome resulting in strains: Δupp ΔCTK_C27600::pnifJ-C. perfingenes glutamate decarboxylase, Δupp ΔCTK_C27600::pnifJ-C. celluvorans lysine decarboxylase, and Δupp ΔCTK_C27600::pnifJ-C. beijerinkii agmatine deaminase. Work is ongoing to characterize the strains.

The foregoing disclosure has been set forth merely to illustrate the invention and is not intended to be limiting.

We claim:

1. A non-naturally occurring Clostridium sp. organism exhibiting increased growth and production of butyric acid at pHs lower than 7.0 when compared to a wild type Clostridium sp. in the same conditions.

2. The non-naturally occurring Clostridium sp. organism of claim 1 exhibiting increased growth and production of butyric acid at pHs lower than 6.5, 5.0 or 4.5 when compared to a wild type Clostridium sp. in the same conditions.

3. The non-naturally occurring Clostridium sp. organism of claim 1 wherein the organism is derived from Clostridium tyrobutyricum (WT ATCC25795) and comprises at least one non-naturally occurring Clostridium tyrobutyricum (WT ATCC25795) gene selected from the group consisting of CTK_RS02840, CTK_RS02785, CTK_RS09915, CTK_RS08580, CTK_RS08585, CTK_RS13265, GABEKIKG_03101, CTK_RS11850, CTK_RS11005, GABEKIKG_01456, spoVD_3, and CTK_RS10285.

4. The non-naturally occurring Clostridium sp. organism of claim 1 wherein the organism is derived from Clostridium tyrobutyricum (WT ATCC25795) and comprises at least one non-naturally occurring Clostridium tyrobutyricum (WT ATCC25795) gene selected from the group consisting of CTK_RS02860, CTK_RS09915, CTK_RS10080, argJ, GABEKIKG_02925, CTK_RS13265, GABEKIKG_03101, CTK_RS11850, CTK_RS11540, GABEKIKG_01456, and CTK_RS10285.

5. The non-naturally occurring Clostridium sp. organism of claim 1 wherein the organism is derived from Clostridium tyrobutyricum (WT ATCC25795) and comprises at least one non-naturally occurring Clostridium tyrobutyricum (WT ATCC25795) gene selected from the group consisting of CTK_RS02840, mob, CTK_RS09785, CTK_RS09915, CTK_RS08710, CTK_RS08580, CTK_RS08585, CTK_RS07220, CTK_RS14515, CTK_RS00560, CTK_RS00210, GABEKIKG_03101, GABEKIKG_00933, apbE, GABEKIKG_01456, spoVD_3, CTK_RS00650, and CTK_RS10290.

6. The non-naturally occurring Clostridium sp. organism of claim 1 wherein the organism is derived from Clostridium tyrobutyricum (WT ATCC25795) and comprises at least one non-naturally occurring Clostridium tyrobutyricum (WT ATCC25795) gene selected from the group consisting of CTK_RS09915, CTK_RS08580, CTK_RS08585, CTK_RS00560, CTK_RS13265, CTK_RS11850, GABEKIKG_01456, spoVD_3, and CTK_RS10285.

7. The non-naturally occurring Clostridium sp. organism of claim 1 wherein the organism comprises at least one heterologous gene encoding for an amino acid decarboxylase or an amino acid deaminase.

8. The non-naturally occurring Clostridium sp. organism of claim 7 comprising at least one heterologous gene encoding for lysine decarboxylase, agmatine deaminase and glutamine decarboxylase.

9. The non-naturally occurring Clostridium sp. organism of claim 8 wherein the at least one heterologous gene encodes for enzymes selected from the group consisting of lysine decarboxylase from C. tyrobutyricum, lysine decarboxylase from Clostridium celluvorans, agmatine deaminase from Clostridium beijerinckii, and glutamine decarboxylase.

10. The non-naturally occurring Clostridium sp. organism of claim 1 wherein the organism generates butyric acid at a rate that is 50% greater than that of a wild type Clostridium sp. organism.

11. The non-naturally occurring Clostridium sp. organism of claim 10 wherein the organism generates butyric acid at a rate of up to 0.48 g/L/h.

12. The non-naturally occurring Clostridium sp. organism of claim 1 wherein the organism expresses at least one heterologous amino acid decarboxylase or heterologous amino acid deaminase.

13. The non-naturally occurring Clostridium sp. organism of claim 12 comprising at least one heterologous amino acid decarboxylase or amino acid deaminase selected from the group consisting of lysine decarboxylase, an agmatine deaminase, and a glutamine decarboxylase.

14. The non-naturally occurring Clostridium sp. organism of claim 13 wherein the at least one heterologous amino acid decarboxylase or amino acid deaminase is selected from the group consisting of lysine decarboxylase from C. tyrobutyricum, a lysine decarboxylase from Clostridium celluvorans, an agmatine deaminase from Clostridium beijerinckii, and a glutamine decarboxylase from Clostridium perfringens.

15. A method for separating carboxylic acids from a fermentation broth comprising growing a non-naturally occurring Clostridium sp. in a fermentation broth with a pH of less than 7.0 and isolating the carboxylic acids from the fermentation broth.

16. The method of claim 15 wherein the growth of the non-naturally occurring Clostridium sp. takes place in a fermentation broth with a pH of less than 6.5, 5.0 or 4.5.

17. The method of claim 15 wherein the non-naturally occurring Clostridium sp. comprises at least one mutation in Clostridium tyrobutyricum (WT ATCC25795) genes selected from the group consisting of CTK_RS02840, CTK_RS02785, CTK_RS09915, CTK_RS08580, CTK_RS08585, CTK_RS13265, GABEKIKG_03101, CTK_RS11850, CTK_RS11005, GABEKIKG_01456, spoVD_3, and CTK_RS10285.

18. The method of claim 15 wherein the non-naturally occurring Clostridium sp. comprises at least one mutation in Clostridium tyrobutyricum (WT ATCC25795) genes selected from the group consisting of CTK_RS02840, mob, CTK_RS09785, CTK_RS09915, CTK_RS08710, CTK_RS08580, CTK_RS08585, CTK_RS07220, CTK_RS14515, CTK_RS00560, CTK_RS00210, GABEKIKG_03101, GABEKIKG_00933, apbE, GABEKIKG_01456, spoVD_3, CTK_RS00650, and CTK_RS10290.

19. The method of claim 15 wherein the non-naturally occurring Clostridium sp. comprises at least one heterologous gene encoding for an amino acid decarboxylase or an amino acid deaminase.

20. The method of claim 15 wherein the non-naturally occurring Clostridium sp. comprises at least one heterologous gene encoding for enzymes selected from the group consisting of lysine decarboxylase from Clostridium tyrobutyricum, lysine decarboxylase from Clostridium celluvorans, agmatine deaminase from Clostridium beijerinckii, and glutamine decarboxylase.
